(19) United States
(12) Patent Application Publication
(10) Pub. No.: US 2026/0035692 A1
(43) Pub. Date: Feb. 5, 2026

(54) METHOD FOR SCREENING FOR APTAMER BY SEQUENCING

(71) Applicant: GeneMind Biosciences Co., Ltd., Shenzhen (CN)

(72) Inventors: Weiwei Luo, Shenzhen (CN); Lei Liu, Shenzhen (CN); Huan Jin, Shenzhen (CN)

(21) Appl. No.: 19/361,870

(22) Filed: Oct. 17, 2025

Related U.S. Application Data
(63) Continuation-in-part of application No. PCT/CN2024/074358, filed on Jan. 29, 2024.

(30) Foreign Application Priority Data
Apr. 21, 2023 (CN) 202310461877.X

(51) Int. Cl. C12N 15/10; C12Q 1/6855; C12Q 1/6869; G01N 21/64

(52) U.S. Cl. CPC C12N 15/1093; C12N 15/1065; C12Q 1/6855; C12Q 1/6869; G01N 21/6428; G01N 21/6458

(57) ABSTRACT

Provided is a method for screening for an aptamer by sequencing. The method comprises: performing sequencing on a plurality of candidate aptamers linked to the surface of a solid-phase carrier to obtain sequencing data; contacting the candidate aptamers with a target in a liquid environment, and coupling with the target any candidate aptamer, among the plurality of candidate aptamers, that has selectivity to the target to obtain a conjugate; detecting a signal generated by the conjugate; and on the basis of the sequencing data and the signal, determining an aptamer that has selectivity with respect to the target. Also provided is a method for screening for an aptamer by sequencing, which can reduce the false positive rate occurring in aptamer screening and increase the throughput of specific aptamers outputted per unit time.

### PRIORITY CLAIM

This application is a (bypass) continuation-in-part of International Application No. PCT/CN2024/074358, filed on Jan. 29, 2024, which claims priority to Chinese Application No. 202310461877.X, filed on Apr. 21, 2023, both of which are hereby incorporated by reference in their entireties.

### SEQUENCE LISTING

The contents in the “Sequence Listing XML” file, named “GMB0040US_SequenceListing_25Sept2025.xml”, created on Sep. 25, 2025, having a file size of 8,443 bytes and filed on even date herewith, is hereby incorporated by reference in its entirety into the subject application.

### TECHNICAL FIELD

The present application relates to the field of molecular biology, and in particular, to a method for screening for an aptamer by sequencing.

### BACKGROUND

Aptamers are oligonucleotide sequences (nucleic acid aptamers) or short polypeptides that are capable of binding with high specificity and high selectivity to various target molecules through conformational changes of their own structures, thereby exhibiting strict recognition ability and high affinity. Currently, aptamers are generally obtained by means of an in vitro screening technology, namely systematic evolution of ligands by exponential enrichment (SELEX). Taking nucleic acid aptamers as an example, the basic principle of SELEX is as follows: by employing molecular biology techniques, a single-stranded oligonucleotide fragment library with random sequences (hereinafter referred to as an oligonucleotide library) is artificially synthesized; the random oligonucleotide library is incubated with a target molecule, and those oligonucleotides bound to the target molecule are screened out. The retained oligonucleotides are then amplified, and the screening step is repeated, such that nucleic acid aptamers capable of specifically binding to a particular target molecule can be obtained. However, screening for nucleic acid aptamers using this method has the following disadvantages: first, since SELEX can only screen for nucleic acid aptamers corresponding to one type of target molecule at a time, the throughput of outputting specific nucleic acid aptamers per unit time is low; and second, the oligonucleotide fragments in the random oligonucleotide library are highly diverse, and a large number of different oligonucleotide fragments may bind to the target molecule, such that the binding ability of the oligonucleotide fragments to the target molecule cannot exhibit a good positive correlation with their binding frequency, thereby resulting in a high false positive rate.

### SUMMARY

The present disclosure provides a method for screening for an aptamer by sequencing, which is intended to at least solve, to some extent, one of the technical problems existing in the prior art or at least provide a useful means.

The inventors made the present disclosure based on the following hypotheses and findings confirmed by experimental verifications:

First, an aptamer is a single-stranded oligonucleotide or a short polypeptide capable of specifically binding to a target molecule. It is inferred that single-stranded nucleic acid molecules or polypeptide molecules having the same sequence as the aptamer, when immobilized on the surface of a solid carrier, are likewise capable of binding to the specific target molecule and can form the three-dimensional structure corresponding to the aptamer.

Second, the ability of aptamer clonal clusters to capture and bind target molecules is related to the copy number of the molecules in the aptamer clonal cluster. The greater the copy number, the stronger the ability of the aptamer clonal clusters to capture and bind the target molecules. Since the aptamer clonal cluster formed on the surface of the solid carrier contains thousands of copies, such aptamer clonal cluster exhibits stronger capture and binding ability toward the target molecule. It can therefore be inferred that the number of conjugates formed between aptamer clonal clusters formed on the surface of the solid carrier and the target molecules is proportional to the copy number of the nucleic acid aptamers on the surface.

Third, the aptamer clonal clusters formed on the surface of the solid carrier can be subjected to in situ sequencing to obtain the sequences of the aptamers. Moreover, since each aptamer clonal cluster formed on the surface of the solid carrier contains thousands of copies, and the aptamers exhibit strong capture and binding ability toward the target molecules, it is inferred that a variety of aptamers can be simultaneously immobilized on the surface of the solid carrier, and a plurality of target molecules can be subjected to in situ screening. Thus, simultaneous detection of multiple aptamer libraries and simultaneous in situ screening against a plurality of target molecules can be achieved at a time, thereby improving the screening throughput and the accuracy of the screening results for aptamers.

The present disclosure provides a method for screening for an aptamer by sequencing. The method includes:
(a) sequencing a plurality of candidate aptamers attached to the surface of a solid carrier to obtain sequencing data;
(b) contacting the candidate aptamers with a target in a liquid environment, and allowing any of the candidate aptamers thereon having selectivity for the target to conjugate with the target to obtain a conjugate;
(c) detecting a signal generated by the conjugate; and
(d) determining an aptamer having selectivity for the target based on the sequencing data and the signal.

According to the method for screening for an aptamer by sequencing provided in the present application, after in situ sequencing is performed on a plurality of candidate aptamers attached onto the surface of the solid carrier, in situ screening is performed on the candidate aptamers on the surface of the solid carrier by using a target. The method is capable of presenting differences in the binding ability between the target and the aptamers, thereby establishing a favorable positive correlation between the binding ability and the binding frequency of the aptamers to the target. As a result, the accuracy of aptamer screening is improved, and the false positive rate in aptamer screening is reduced. In addition, the method does not require repeated cycles of aptamer screening. Through a single in situ sequencing-in situ screening process, a plurality of candidate aptamers and a plurality of targets can be screened simultaneously. The method not only simplifies the aptamer screening process and reduces the time cost, but also improves the screening efficiency, thereby increasing the throughput of outputting specific aptamers per unit time.

Additional aspects and advantages of the embodiments of the present disclosure will be partially set forth in the following description, and will partially become apparent from the following description or be appreciated by practice of the embodiments of the present disclosure.

### BRIEF DESCRIPTION OF THE DRAWINGS

To more clearly illustrate the technical solutions in the embodiments of the present application or in the prior art, the drawings required for use in the description of the embodiments or the prior art will be briefly described below. It is obvious that the drawings in the description below are only some embodiments of the present application, and other drawings can be derived from the drawings by those of ordinary skill in the art without creative efforts.

Among the drawings:

FIG. 1 is a fluorescence image obtained by imaging according to a verification example of the present application;

FIG. 2 is a fluorescence image obtained by imaging according to Example 1 of the present application;

FIG. 3 is a fluorescence image obtained by imaging according to Example 2 of the present application;

FIG. 4 is a combined fluorescence image of FIG. 3; and

FIG. 5 is a fluorescence image obtained in Example 2 of the present application after labeling fluorescence signals.

### DETAILED DESCRIPTION

The technical solutions of the embodiments of the present application will be clearly and completely described below with reference to the drawings of the embodiments of the present application, and apparently, the described embodiments are only a part of the embodiments of the present application instead of all embodiments of the present application. On the basis of the embodiments of the present application, all other embodiments obtained by those of ordinary skill in the art without creative efforts shall fall within the claimed scope of the present application.

In the embodiments of the present disclosure, it should be understood that the terms “first” and “second” are used for description purpose only rather than being construed as indicating or implying relative importance or implicitly indicating the number of indicated technical features; the features defined with “first” and “second” may explicitly or implicitly include one or more of the features. Unless otherwise specified, “a group” or “a plurality of” means two or more.

It should be noted that, unless otherwise specified, “attaching” and “linking” should be understood in a broad sense. For example, it may be a chemical ligation or a physical connection. For those of ordinary skill in the art, the specific meanings of the aforementioned terms in corresponding examples can be understood according to specific conditions.

Reference numerals and/or letters may be repeatedly used in different examples in the present disclosure for simplicity and clarity rather than for indicating the relationship between various embodiments and/or settings discussed.

In the embodiments of the present application, the term “sequencing” may also be referred to as “nucleic acid sequencing” or “gene sequencing”. The three are used interchangeably and refer to the determination of the type and order of bases or nucleotides (including nucleotide analogs) in a nucleic acid molecule. The sequencing involves the process of binding nucleotides to a template and collecting the corresponding signals emitted by the nucleotides (including analogs). The sequencing includes sequencing by synthesis (SBS) and/or sequencing by ligation (SBL), including DNA sequencing and/or RNA sequencing.

The term “nucleic acid molecule” refers to polymeric forms of nucleotides of any length and may include ribonucleotides or analogs thereof, deoxyribonucleotides or analogs thereof, and mixtures of the above nucleotides or analogs thereof. The nucleic acid molecule may refer to a single-stranded polynucleotide or a double-stranded polynucleotide. Nucleotides in a nucleic acid molecule may include naturally occurring nucleotides and functionally alternative analogs thereof. Examples of analogs can hybridize to nucleic acids in a sequence-specific manner, or can be used as templates for the replication of particular nucleotide sequences. Naturally occurring nucleotides generally have a backbone containing a phosphodiester bond. Analog structures may have alternative backbone linkages including any types known in the art. Naturally occurring nucleotides generally have deoxyribose (e.g., found in DNA) or ribose (e.g., found in RNA). Analog structures may have alternative sugar moieties including any types known in the art. Nucleotides may contain natural bases or non-natural bases. Bases in natural DNA may include one or more of adenine, thymine, cytosine, and/or guanine, and bases in natural RNA may include one or more of adenine, uracil, cytosine, and/or guanine. Any non-natural base or base analog may also be used in a nucleotide, such as a locked nucleic acid (LNA) and a bridged nucleic acid (BNA).

The term “clonal cluster”, also referred to as an “amplification cluster”, refers to an aggregate of a plurality of nucleic acid molecules having identical sequences formed at a specific position on a solid carrier, such as a flowcell. The plurality of nucleic acid molecules are derived from the same nucleic acid molecule. For example, the clonal cluster is an amplification cluster of a plurality of nucleic acid molecules formed through amplification of one nucleic acid molecule by bridge PCR.

The term “primer”, also known as “probe”, refers to an oligonucleotide or a nucleic acid molecule that can hybridize to a target sequence of interest. In the embodiments, the primer serves as a substrate onto which nucleotides may be polymerized by a polymerase. For example, the primer may be used as a starting point for DNA or RNA synthesis. For example, a sequencing primer can hybridize to a synthesized nucleic acid template strand so as to trigger the synthesis of a new strand complementary to the synthesized nucleic acid template strand. The primer may include any combination of nucleotides or analogs thereof. In some examples, the primer is a single-stranded oligonucleotide or a polynucleotide.

Aptamers, due to their characteristic ability to bind with high specificity and high selectivity to various target molecules (such as proteins, viruses, bacteria, cells, and heavy metal ions), possess broad application prospects in fields including cell imaging, new drug research and development, disease therapy, microbial detection, and the like. Screening for an aptamer from numerous candidate aptamers is a critical step that enables the application of the aptamer.

Taking nucleic acid aptamers as an example, as an existing aptamer screening method, SELEX can only screen for nucleic acid aptamers corresponding to one type of target at a time, the throughput of outputting specific nucleic acid aptamers per unit time is consequently low; and moreover, since the oligonucleotide fragments in the random oligonucleotide library are highly diverse, and a large number of different oligonucleotide fragments may bind to the target molecule, the binding ability of the oligonucleotide fragments to the target molecule cannot exhibit a good positive correlation with their binding frequency, thereby resulting in a high false positive rate. Therefore, reducing the false positive rate and improving the screening efficiency of nucleic acid aptamers remain significant challenges in aptamer screening.

To address the above problems, the present application provides a method for screening for an aptamer, which enables synchronous screening of a plurality of candidate aptamers having sequencing results and a plurality of targets in a single process. Moreover, the binding ability of an aptamer to a target molecule demonstrates a favorable positive correlation with its binding frequency, thereby contributing to the reduction of the false positive rate in aptamer screening.

The method for screening for an aptamer by sequencing according to the embodiments of the present application includes:

(a) sequencing a plurality of candidate aptamers attached to the surface of a solid carrier to obtain sequencing data.

In this step, the candidate aptamers refer to aptamers to be screened. The type of aptamer may be a single-stranded nucleic acid molecule, namely a nucleic acid aptamer, or may be a short polypeptide, such as a polypeptide composed of 2-10 amino acids, and more preferably a polypeptide composed of 2-6 amino acids.

In one embodiment, the candidate aptamers are candidate nucleic acid aptamers. The oligonucleotide type of the nucleic acid aptamers may be a single-stranded DNA (deoxyribonucleic acid) sequence, a single-stranded RNA (ribonucleic acid) sequence, or a single-stranded XNA (xeno nucleic acid). In some embodiments, the length of the candidate nucleic acid aptamers is 20-150 nt. The length of the candidate nucleic acid aptamers, as referred to herein, means the number of nucleotides or analogs thereof or the number of bases or analogs thereof constituting the candidate aptamers. It should be understood that the lengths of the plurality of candidate aptamers all fall within 20-150 nt, but are not required to be the same. For example, among the plurality of candidate aptamers, there may be a candidate aptamer having a length of 50 nt, a candidate aptamer having a length of 80 nt, a candidate aptamer having a length of 145 nt, or the like, without strict limitations on the length of the individual candidate aptamers. In some embodiments, the length of the candidate nucleic acid aptamers is 20-100 nt.

In the embodiments of the present application, attaching the plurality of candidate aptamers to the surface of the solid carrier refers to attaching the candidate aptamers to the solid carrier by chemical or physical means, thereby immobilizing the candidate aptamers at respective positions on the surface of the solid carrier. That is, each of the candidate aptamers or each clonal cluster formed by the candidate aptamers on the surface of the solid carrier occupies a defined position. Accordingly, the sequence of the candidate aptamer corresponding to each position can be determined by sequencing. After screening and obtaining an aptamer having a selective binding tendency to a specific target, the sequence of the aptamer can be determined based on its position, thereby identifying the relationship between the target and the aptamer sequence. According to the embodiments of the present application, before attaching the plurality of candidate aptamers to the surface of the solid carrier, the method further includes constructing a library of candidate aptamers (corresponding to the sequencing library described below).

As one embodiment of the embodiments of the present application, the plurality of candidate aptamers attached to the surface of the solid carrier each form a clonal cluster on the surface of the solid carrier, that is, each of the candidate aptamers attached to the solid carrier is immobilized in the form of a clonal cluster at different positions on the surface of the solid carrier. By forming clonal clusters, the copy number of candidate aptamers at each position of the solid carrier can be significantly increased. Accordingly, in one aspect, during the process of screening candidate aptamers in the presence of a target, the clonal clusters enhance the correlation between the binding ability and the binding frequency of the candidate aptamers at each position with the target. Furthermore, by superimposing the correlation across different positions of the same candidate aptamer on the surface of the solid carrier, the binding ability of the candidate aptamer to the target demonstrates a favorable positive correlation with its binding frequency. In another aspect, for the same candidate aptamer, it may bind to different targets, but the binding ability of the same candidate aptamer to different targets exhibits a certain degree of variability. When the candidate aptamer appears in the form of a clonal cluster, the difference in binding ability to different targets is amplified as the copy number of the candidate aptamer increases, thereby facilitating the screening for an aptamer having a binding advantage to a particular target. According to the embodiments of the present application, the method for screening for an aptamer by sequencing further includes, before sequencing: amplifying the candidate aptamers to obtain clonal clusters.

In an embodiment of the present application, (a) includes:
(a-10) obtaining a sequencing library including the candidate aptamers;
(a-30) attaching the sequencing library onto the surface of the solid carrier;
(a-50) amplifying the sequencing library on the surface of the solid carrier to obtain a clonal cluster; and
(a-70) sequencing the clonal cluster to obtain the sequencing data.

In step (a-10), obtaining the sequencing library including the candidate aptamers refers to obtaining a sequencing library including a plurality of candidate aptamers. The manner of obtaining the sequencing library is not strictly limited in the embodiments of the present application. In some embodiments, (a-10) includes: separately constructing a sequencing library for each of the candidate aptamers; and mixing sequencing libraries of the plurality of candidate aptamers according to a predetermined ratio. In this method, the type of candidate aptamers included in the sequencing library is not strictly limited. In theory, the larger the surface of the solid carrier used for immobilizing candidate aptamers, the more types of candidate aptamers may be immobilized. When a plurality of candidate aptamers are mixed, the proportion of each candidate aptamer may be configured according to a predetermined ratio, and the predetermined ratio includes a random ratio. In the embodiments of the present application, the type of the predetermined ratio may be the relative content or absolute content of the sequencing library, and the content can be expressed by means such as concentration or volume. It should be understood that when the sequencing libraries of a plurality of candidate aptamers are mixed, the same content parameter is selected, and the candidate aptamers are mixed according to the predetermined ratio.

In an embodiment, both ends of each of the candidate aptamers have common sequences, which provide primer binding sites for generating a complementary strand of the candidate aptamer. The common sequences referred to herein refer to stretches of oligonucleotides having the same sequence. In this case, separately constructing the sequencing library for each of the candidate aptamers includes: converting the candidate aptamers into double-stranded nucleic acid molecules by using the common sequences, such that the ends of the double-stranded nucleic acid molecules have specific tags and adapters. One implementation of converting the candidate aptamers into double-stranded nucleic acid molecules by using the common sequences may be: using the common sequence at one end of the candidate aptamer as a primer binding site, and generating a complementary strand of the candidate aptamer based on the principle of base complementarity. Then, the ends of the double-stranded nucleic acid molecules are provided with specific tags and adapters. The specific tag is used to distinguish different candidate aptamers, that is, the same candidate aptamer carries the same specific tag, while different candidate aptamers carry different specific tags. The adapter is used to attach to the probe on the surface of the solid carrier, thereby enabling the candidate aptamers to be attached to the surface of the solid carrier. In one possible implementation, the adapter may be the common sequence of the candidate aptamer, that is, the common sequence serves as the adapter; in another possible implementation, the common sequence serves as a portion of the adapter.

In an embodiment, after the candidate aptamers are converted into double-stranded nucleic acid molecules by using the common sequences, the method further includes amplifying the double-stranded nucleic acid molecules to increase the copy number of the double-stranded nucleic acid molecules, i.e., to increase the copy number of the candidate aptamers. Correspondingly, in the step of attaching specific tags and adapters to the ends of the double-stranded nucleic acid molecules, the double-stranded nucleic acid molecules include both the double-stranded nucleic acid molecules obtained by converting the candidate aptamers using the common sequences and the double-stranded nucleic acid molecules obtained by amplification. That is, both ends of the candidate aptamer have common sequences. Separately constructing the sequencing library for each of the candidate aptamers includes: converting the candidate aptamers into double-stranded nucleic acid molecules by using the common sequences, and then amplifying the double-stranded nucleic acid molecules, such that the ends of the amplified double-stranded nucleic acid molecules have specific tags and adapters.

In some embodiments, sequence diversity is improved and base balance during sequencing is achieved by adding a balance library into the sequencing library. Correspondingly, (a-10) further includes: making the sequencing library include a balance library. The balance library is selected from reference libraries having reference standards, and the balance library does not bind to the target. In some embodiments, the balance library is selected from microbial libraries having a GC content of 40%-50%, thereby providing favorable base diversity and reference value. Illustratively, the balance library is selected from at least one of E. coli 8 and PhiX174. In this embodiment, the proportion of the balance library is not less than 10% of the total content of the sequencing library, where the proportion herein refers to content proportion. Similarly, the content may be either relative content or absolute content, and both the relative content and the absolute content can be expressed by means such as concentration and volume.

In some embodiments, the sequencing library may be denatured after (a-10) or before (a-30) to obtain a single-stranded sequencing library. In the single-stranded sequencing library, except for the single-stranded balance library, other single-stranded sequencing libraries constitute the library of candidate aptamers.

Step (a-30) involves attaching the sequencing library onto the surface of the solid carrier to immobilize the sequencing library on the surface of the solid carrier. In some embodiments, the sequencing library is linked to the probe on the surface of the solid carrier via library molecules such as adapters of the candidate aptamers so as to immobilize the sequencing library on the surface of the solid carrier. In one embodiment, the sequencing library is linked through a polymerization reaction between the terminal nucleotides of the adapter and the terminal nucleotides of the probe on the surface of the solid carrier. In another embodiment, the sequencing library is linked by base pairing through hydrogen bonds between at least a portion of the adapter and the probe on the surface of the solid carrier. Compared to the first embodiment, the second embodiment is more advantageous in stably immobilizing the candidate aptamers on the surface of the solid carrier, thereby facilitating the screening of aptamers.

It should be understood that when the sequencing library is a single-stranded sequencing library, correspondingly, the sequencing library immobilized on the surface of the solid carrier is a single-stranded sequencing library. When the sequencing library is a double-stranded sequencing library, correspondingly, the sequencing library immobilized on the surface of the solid carrier is a double-stranded sequencing library. If the sequencing library is a double-stranded sequencing library, after (a-30) or before (a-50), the method further includes: denaturing the sequencing library to obtain a single-stranded sequencing library.

In step (a-50), the sequencing library is amplified to obtain a clonal cluster. Since the sequencing library is attached onto the surface of the solid carrier, the amplification in step (a-50) is performed on the surface of the solid carrier. In some embodiments, the amplification is selected from at least one of bridge amplification and template-walking amplification. In an embodiment, the amplification is isothermal amplification.

In step (a-70), the sequencing data of the library molecules immobilized at various positions on the solid carrier is obtained by sequencing the clonal cluster, where the library molecules include candidate aptamers, and when the sequencing library includes a balance library, the library molecules further include balance library molecules.

In some embodiments, the sequencing is sequencing by synthesis, which includes: introducing a fluid containing a sequencing primer onto the surface of the solid carrier and subjecting the surface of the solid carrier to conditions suitable for a polymerization reaction, so as to controllably extend at least a portion of the sequencing primer and correspondingly detect an extension signal, where the sequencing primer is capable of matching at least a portion of the adapter. In an embodiment, the sequencing by synthesis includes the following steps.

In (a-71), a sequencing primer is bound at the adapter, where the sequencing primer matches at least a portion of the adapter.

This step includes: introducing a fluid containing a sequencing primer onto the surface of the solid carrier, where the sequencing primer matches the adapter on the candidate aptamer, such that the sequencing primer is complementarily paired with the adapter to bind to the candidate aptamer. It should be understood that when the sequencing library includes a balance library, the sequencing primer also matches the adapter of the balance library, such that the sequencing primer is complementarily paired with the adapter of the balance library to bind to the balance library.

In (a-72), the sequencing primer is subjected to base extension.

This step is performed under conditions suitable for a nucleotide polymerization reaction, under which the added dNTPs are sequentially bound to the sequencing primer according to the principle of base complementarity, thereby extending the nucleic acid molecule strand containing the sequencing primer. It should be understood that each cycle of the base extension is expected to incorporate one nucleotide into the nucleic acid molecule strand containing the sequencing primer, i.e., to increase the length of the nucleic acid molecule containing the sequencing primer by one nucleotide. However, in some cases, two or more nucleotides may be incorporated into the nucleic acid molecule strand containing the sequencing primer in one cycle of base extension. Such an event is rare during the base extension, and does not affect the overall process of the base extension, but may cause certain interference with the sequencing signals generated during the base extension, which needs to be eliminated by using appropriate correction means.

In (a-73), sequencing signals generated in each cycle of base extension are detected.

The acquisition of sequencing signals generated after each cycle of base extension in step (a-73) may refer to acquiring detectable signals carried by the nucleotide incorporated into the nucleic acid molecule containing the sequencing primer in each cycle of base extension, or acquiring detectable signals generated during the incorporation of the nucleotide into the nucleic acid molecule containing the sequencing primer in each cycle of base extension. The type of the detectable signal is not strictly limited. Illustratively, the detectable signal is a fluorescence signal, and correspondingly, the sequencing signal is a fluorescence signal.

In (a-74), the sequencing data of the candidate aptamers are determined based on the sequencing signals.

In this step, the sequencing data referred to is data from which the sequences of the candidate aptamers can be directly obtained, such as sequencing data in the form of sequencing reads, or data from which the sequences of the candidate aptamers can be derived, such as fluorescence images. Illustratively, the sequencing data may be raw data obtained from sequencing, such as initial fluorescence images obtained based on sequencing by synthesis, or may be secondary data obtained after processing the raw data obtained from sequencing, such as corrected fluorescence images based on the initial fluorescence images obtained from sequencing by synthesis, or sequencing reads of the candidate aptamers obtained from sequencing.

Illustratively, in (a-70), the sequencing is sequencing by synthesis based on microscopic fluorescence imaging, performing corresponding extension signal detection includes exciting the fluorescent label on the surface of the solid carrier after extension to emit light and capturing images, and obtaining the sequencing data includes: constructing a template based on a plurality of images obtained by capturing images, where the template is a set of specific light-emitting positions on the plurality of images, and the set of specific light-emitting positions correspond to positional information of the plurality of candidate aptamers on the surface of the solid carrier; identifying the types of extended bases based on the images and the template to obtain a plurality of reads; and demultiplexing the reads based on portions of the reads corresponding to the specific tags to obtain the sequencing data of each of the candidate aptamers.

In another embodiment of the present application, (a) includes:
(a-20) obtaining a sequencing library including the candidate aptamers, the sequencing library being a circular single-stranded nucleic acid molecule with an adapter;
(a-40) performing rolling circle amplification on the sequencing library to form nanoballs;
(a-60) attaching the nanoballs onto the surface of the solid carrier; and
(a-80) sequencing the nanoballs to obtain the sequencing data.

In step (a-20), obtaining the sequencing library including the candidate aptamers refers to obtaining a sequencing library including a plurality of candidate aptamers, and the sequencing library is a circular single-stranded nucleic acid molecule with an adapter. The manner of obtaining the sequencing library is not strictly limited in the embodiments of the present application. Step (a-20) includes: separately constructing a sequencing library for each of the candidate aptamers. In one embodiment, separately constructing the sequencing library for each of the candidate aptamers includes: making the sequencing library for each of the candidate aptamers have a specific tag and an adapter. The specific tag is used to distinguish different candidate aptamers, that is, the same candidate aptamer carries the same specific tag, while different candidate aptamers carry different specific tags. The adapter is used to attach to the probe on the surface of the solid carrier, thereby enabling the candidate aptamers to be attached to the surface of the solid carrier.

In some embodiments, step (a-20) further includes: mixing sequencing libraries of the plurality of candidate aptamers according to a predetermined ratio. In this method, the type of candidate aptamers included in the sequencing library is not strictly limited. In theory, the larger the surface of the solid carrier used for immobilizing candidate aptamers, the more types of candidate aptamers may be immobilized. When a plurality of candidate aptamers are mixed, the proportion of each candidate aptamer may be configured according to a predetermined ratio, and the predetermined ratio includes a random ratio. In the embodiments of the present application, the type of the predetermined ratio may be the relative content or absolute content of the sequencing library, and the content can be expressed by means such as concentration or volume. It should be understood that when the sequencing libraries of a plurality of candidate aptamers are mixed, the same content parameter is selected, and the candidate aptamers are mixed according to the predetermined ratio.

In some embodiments, sequence diversity is improved and base balance during sequencing is achieved by adding a balance library into the sequencing library. Correspondingly, (a-20) further includes: making the sequencing library include a balance library. Similarly, the balance library is a circular single-stranded nucleic acid molecule with an adapter. The balance library is selected from reference libraries having reference standards. In some embodiments, the balance library is selected from microbial libraries having a GC content of 40%-50%, thereby providing favorable base diversity and reference value. Illustratively, the balance library is selected from at least one of E. coli 8 and PhiX174. In this embodiment, the proportion of the balance library is not less than 10% of the total content of the sequencing library, where the proportion herein refers to content proportion. Similarly, the content may be either relative content or absolute content, and both the relative content and the absolute content can be expressed by means such as concentration and volume.

In step (a-20), the sequencing library initially provided may be a linear nucleic acid molecule. In this case, the closure of the linear nucleic acid molecule may be achieved by a padlock probe under the action of a ligase.

In step (a-40), the sequencing library is amplified by rolling circle amplification (RCA) to form nanoballs. In this step, a circular nucleic acid molecule serves as the template, and under the catalysis of a polymerase, dNTPs are sequentially polymerized and extended on the primer strand according to the principle of complementary base pairing, thereby forming a single-stranded nucleic acid molecule including hundreds to thousands of repeated template-complementary fragments.

In some embodiments, when step (a-20) includes separately constructing a sequencing library for each of the candidate aptamers, step (a-40) includes (a-42): performing rolling circle amplification on the sequencing library for each of the candidate aptamers to form nanoballs of each of the candidate aptamers; and (a-43): mixing the nanoballs of the plurality of candidate aptamers according to a predetermined ratio. In step (a-43), the type of candidate aptamers included in the sequencing library is not strictly limited. In theory, the larger the surface of the solid carrier used for immobilizing candidate aptamers, the more types of candidate aptamers may be immobilized. When the nanoballs of a plurality of candidate aptamers are mixed, the proportion of each candidate aptamer may be configured according to a predetermined ratio, and the predetermined ratio includes a random ratio. In the embodiments of the present application, the type of the predetermined ratio may be the relative content or absolute content of the sequencing library, and the content can be expressed by means such as concentration or volume. It should be understood that when the sequencing libraries of a plurality of candidate aptamers are mixed, the same content parameter is selected, and the candidate aptamers are mixed according to the predetermined ratio.

In some embodiments, when step (a-20) includes: separately constructing a sequencing library for each of the candidate aptamers, and mixing sequencing libraries of the plurality of candidate aptamers according to a predetermined ratio, step (a-40) includes: performing rolling circle amplification on the sequencing library for each of the candidate aptamers to form nanoballs.

Step (a-60) involves attaching the nanoballs onto the surface of the solid carrier to immobilize the nanoballs on the surface of the solid carrier. When step (a-40) includes (a-42) and (a-43), step (a-60) is (a-62): attaching the mixed nanoballs onto the surface of the solid carrier. In some embodiments, the nanoballs are linked to the probe on the surface of the solid carrier via an adapter so as to immobilize the nanoballs on the surface of the solid carrier. In one embodiment, the nanoballs are linked through a polymerization reaction between the terminal nucleotides of the adapter and the terminal nucleotides of the probe on the surface of the solid carrier. In another embodiment, the nanoballs are linked by base pairing through hydrogen bonds between at least a portion of the adapter and the probe on the surface of the solid carrier. Compared to the first embodiment, the second embodiment is more advantageous in stably immobilizing the nanoballs on the surface of the solid carrier, thereby facilitating the screening of aptamers.

In step (a-70), the sequencing data of the nanoballs immobilized at various positions on the solid carrier is obtained by sequencing the nanoballs, where the nanoballs include the sequences of candidate aptamers, and when the sequencing library includes a balance library, the library molecules further include the sequences of balance library molecules.

In some embodiments, the sequencing is sequencing by synthesis, which includes: introducing a fluid containing a sequencing primer onto the surface of the solid carrier and subjecting the surface of the solid carrier to conditions suitable for a polymerization reaction, so as to controllably extend at least a portion of the sequencing primer and correspondingly detect an extension signal, where the sequencing primer is capable of matching at least a portion of the adapter. In an embodiment, the sequencing by synthesis includes the following steps.

In (a-81), a sequencing primer is bound at the adapter, where the sequencing primer matches at least a portion of the adapter.

This step includes: introducing a fluid containing a sequencing primer onto the surface of the solid carrier, where the sequencing primer matches the adapter on the candidate aptamer, such that the sequencing primer is complementarily paired with the adapter to bind to the candidate aptamer. It should be understood that when the sequencing library includes a balance library, the sequencing primer also matches the adapter of the balance library, such that the sequencing primer is complementarily paired with the adapter of the balance library to bind to the balance library.

In (a-82), the sequencing primer is subjected to base extension.

This step is performed under conditions suitable for a nucleotide polymerization reaction, under which the added dNTPs are sequentially bound to the sequencing primer according to the principle of base complementarity, thereby extending the nucleic acid molecule strand containing the sequencing primer. It should be understood that each cycle of the base extension is expected to incorporate one nucleotide into the nucleic acid molecule strand containing the sequencing primer, i.e., to increase the length of the nucleic acid molecule containing the sequencing primer by one nucleotide. However, in some cases, two or more nucleotides may be incorporated into the nucleic acid molecule strand containing the sequencing primer in one cycle of base extension. Such an event is rare during the base extension, and does not affect the overall process of the base extension, but may cause certain interference with the sequencing signals generated during the base extension, which needs to be eliminated by using appropriate correction means.

In (a-83), sequencing signals generated in each cycle of base extension are detected.

The acquisition of sequencing signals generated after each cycle of base extension in step (a-83) may refer to acquiring detectable signals carried by the nucleotide incorporated into the nucleic acid molecule containing the sequencing primer in each cycle of base extension, or acquiring detectable signals generated during the incorporation of the nucleotide into the nucleic acid molecule containing the sequencing primer in each cycle of base extension. The type of the detectable signal is not strictly limited. Illustratively, the detectable signal is a fluorescence signal, and correspondingly, the sequencing signal is a fluorescence signal.

In (a-84), the sequencing data of the candidate aptamers are determined based on the sequencing signals.

In this step, the sequencing data referred to is data from which the sequences of the candidate aptamers can be directly obtained, such as sequencing data in the form of sequencing reads, or data from which the sequences of the candidate aptamers can be derived, such as fluorescence images. Illustratively, the sequencing data may be raw data obtained from sequencing, such as initial fluorescence images obtained based on sequencing by synthesis, or may be secondary data obtained after processing the raw data obtained from sequencing, such as corrected fluorescence images based on the initial fluorescence images obtained from sequencing by synthesis, or sequencing reads of the candidate aptamers obtained from sequencing.

Illustratively, in (a-70), the sequencing is sequencing by synthesis based on microscopic fluorescence imaging, performing corresponding extension signal detection includes exciting the fluorescent label on the surface of the solid carrier after extension to emit light and capturing images, and obtaining the sequencing data includes: constructing a template based on a plurality of images obtained by capturing images, where the template is a set of specific light-emitting positions on the plurality of images, and the set of specific light-emitting positions correspond to positional information of the plurality of candidate aptamers on the surface of the solid carrier; identifying the types of extended bases based on the images and the template to obtain a plurality of reads; and demultiplexing the reads based on portions of the reads corresponding to the specific tags to obtain the sequencing data of each of the candidate aptamers.

In an embodiment, (a) includes:
(a-22) separately constructing a sequencing library for each of the candidate aptamers, including making the sequencing library for each of the candidate aptamers have a specific tag and an adapter;
(a-42) performing rolling circle amplification on the sequencing libraries for each of the candidate aptamers to form nanoballs of each of the candidate aptamers;
(a-43) mixing the nanoballs of the plurality of candidate aptamers according to a predetermined ratio;
(a-62) attaching the mixed nanoballs onto the surface of the solid carrier; and
(a-80) sequencing the nanoballs to obtain the sequencing data.

The implementation of the above steps is as described above, and will not be recited here for brevity.

The method for screening for an aptamer by sequencing according to the embodiments of the present application further includes:
(b) contacting the candidate aptamers with a target in a liquid environment, and allowing any of the candidate aptamers thereon having selectivity for the target to conjugate with the target to obtain a conjugate.

In step (b), conjugates are obtained by introducing the target to enable conjugation between candidate aptamers and the target that have mutual selectivity. In some embodiments, when the candidate aptamers on the solid carrier are bound with a sequencing primer or with an extended strand formed by the sequencing primer, the method for screening for an aptamer provided in the present application further includes: removing the sequencing primer or an extended strand formed by the sequencing primer before (b), so as to eliminate interference of the sequencing primer or the extended strand formed by the sequencing primer with the binding site of the target.

In the embodiments of the present application, contacting the candidate aptamers on the surface of the solid carrier with the target is performed in a liquid environment. In one embodiment, the conjugation of the candidate aptamers with the target is achieved by contacting the solid carrier with a target-containing fluid, illustratively, by flowing the target-containing fluid over the surface of the solid carrier. In an embodiment, (b) includes: (b-20) introducing a target-containing fluid onto the surface of the solid carrier after (a), where the target-containing fluid includes one or more types of targets; and (b-40) subjecting the surface of the solid carrier to a designated environment for a certain duration to allow any of the candidate aptamers thereon having selectivity for the target to conjugate with the target to obtain the conjugate. In some embodiments, the concentration of the target in a target-containing solution is not less than 30 nmol/L, so as to enrich detectable signals generated by the conjugation reaction, thereby facilitating the capture of the detectable signals. Within this range, the concentration of the target varies with the binding affinity between the target and the nucleic acid aptamer: the stronger the binding affinity, the lower the required concentration of the target; conversely, the weaker the binding affinity, the higher the required concentration of the target.

In another embodiment, the solid carrier may also be placed in a fluid environment, such as by immersing the solid carrier in a fluid and then adding the target into the fluid, thereby achieving conjugation of the candidate aptamers with the target. In some embodiments, the concentration of the target in a target-containing fluid is not less than 30 nmol/L, so as to enrich detectable signals generated by the conjugation reaction, thereby facilitating the capture of the detectable signals.

In some implementations of the above two embodiments, the target carries a detectable label, such that the conjugate obtained after conjugation between the target and the candidate aptamers bears the detectable label. Illustratively, the detectable label is a fluorophore. When the target-containing fluid or solution includes a plurality of targets, a signal generated by a detectable label carried by any one type of the targets is distinguishable from a signal generated by a detectable label carried by other targets. Illustratively, the plurality of targets all carry fluorophores, and the fluorescence signals generated by the fluorophores carried by the plurality of targets can be distinguished from each other. For example, the fluorescence signals generated by the fluorophores carried by different targets have different colors, such that the fluorescence signals generated by the fluorophores carried by various targets can be directly distinguished; or different fluorophores require different excitation wavelengths, thereby enabling differentiation of the fluorescence signals generated by the fluorophores carried by various targets through controlling detection conditions.

In some other embodiments, detectable signals are generated during the process of conjugation of the candidate aptamers with the target to form a conjugate. When the target-containing fluid or solution includes a plurality of targets, the detectable signals generated when any one type of the targets conjugates with the candidate aptamers are distinguishable from the signals generated when other targets conjugate with the candidate aptamers.

In some embodiments, among the plurality of targets bound to the candidate aptamers, some targets carry detectable labels such that the conjugates obtained after conjugation between the targets and the candidate aptamers bear the detectable labels, while some targets generate detectable signals during the conjugation process with the candidate aptamers to form conjugates. Regardless of whether the targets carry detectable labels or generate detectable signals during conjugation with the candidate aptamers, the detectable signals possessed by the conjugates formed by any one type of the targets with the candidate aptamers are distinguishable from the signals possessed by the conjugates formed by other targets with the candidate aptamers.

In the embodiments of the present application, the type of the target may be selected from at least one of proteins, viruses, bacteria, cells, and heavy metal ions. In one process of screening for an aptamer by sequencing, a plurality of targets of the same type may be added, for example, the plurality of targets all being proteins, or the plurality of targets all being viruses or bacteria, to screen for a corresponding aptamer; alternatively, a plurality of different types of targets may be added, for example, targets containing proteins, viruses, and bacteria simultaneously, to screen for a corresponding aptamer. That is, either the same targets or a combination of different types of targets may be employed in one process of screening for an aptamer by sequencing. In some embodiments, the plurality of targets contained in the target-containing solution or fluid are selected from at least two of streptavidin (interleukin 3 receptor a), colony stimulating factor (CSF), PDGF-BB (recombinant human PDGF-BB or human platelet-derived growth factor BB), mucin 1 (MUC1, CB Number: CB95082617), and human epidermal growth factor receptor 2 (HER2).

The method for screening for an aptamer by sequencing according to the embodiments of the present application further includes:
(c) detecting a signal generated by the conjugate.

In step (c), the signal generated by the conjugate is detected to determine the candidate aptamer that has undergone conjugation. In some embodiments, the target itself carries a detectable label, and after conjugation of the target with the candidate aptamers, the conjugate thus formed carries a detectable label. In step (c), detecting a signal generated by the conjugate refers to detecting the signal generated by the detectable label within the conjugate. Illustratively, the detectable label is a fluorophore. In this case, step (c) may include exciting the fluorophore to emit light and imaging the surface of the solid carrier to collect emission signals. At this time, positions on the surface of the solid carrier containing fluorophores generate emission signals, which, upon identification and imaging, can form a fluorescence image.

Step (b) may enable one or more types of targets to contact with the surface of the solid carrier, thereby allowing one or more types of targets to conjugate with the candidate aptamers to form conjugates. When the target-containing solution or fluid in step (b) includes a plurality of targets, the plurality of targets each carry different fluorophores, such that the fluorescence signals generated by the fluorophores carried by any one type of the targets are distinguishable from the fluorescence signals generated by the fluorophores carried by other targets. Illustratively, different fluorophores require different excitation wavelengths, such that the fluorescence signals generated by fluorophores carried by various targets can be distinguished by controlling detection conditions. In this case, step (c) includes: exciting different fluorophores to emit light separately and imaging the surface of the solid carrier to collect corresponding emission signals.

When the targets introduced in step (b) all undergo conjugation reactions with the candidate aptamers, the signals detected in step (c) are all derived from signals generated by detectable labels carried by the conjugates. If some of the targets introduced in step (b) remain unbound after conjugation reactions with the candidate aptamers, then the signals detected in step (c) include not only the first signal derived from detectable labels carried by the conjugates, but also the second signal derived from detectable labels carried by the free targets. The presence of the second signal interferes with the collection of the first signal. Therefore, the method for screening for an aptamer according to the embodiments of the present application further includes: removing a free target after (b) or before (c), so as to eliminate signal interference caused by detectable labels carried by the free target.

In some embodiments, the target itself does not carry a detectable label, but a detectable signal is generated during the conjugation process between the target and the candidate aptamers. In this case, the signal detected from the conjugate in step (c) is the signal generated during the conjugation process between the target and the candidate aptamers.

Step (b) may enable one or more types of targets to contact with the surface of the solid carrier, thereby allowing one or more types of targets to conjugate with the candidate aptamers to form conjugates. When the target-containing solution or fluid in step (b) includes a plurality of targets, the signals generated by the conjugation between any one type of the targets and the candidate aptamers, and the signals carried by the conjugates formed thereby, are distinguishable from the signals generated when other targets conjugate with the candidate aptamers and the signals carried by the conjugates formed thereby.

In the embodiments of the present application, according to the difference in the number of types of targets introduced at one time in the step of “contacting the candidate aptamers with a target in a liquid environment”, step (b) and step (c) may include two implementations.

In a first implementation, the candidate aptamers are simultaneously contacted with a plurality of targets in one contact process. In this case, step (b) includes: contacting the candidate aptamers with the plurality of targets in a liquid environment, and allowing any of the candidate aptamers thereon having selectivity for the targets to conjugate with the targets to obtain a plurality of conjugates; and step (c) includes: detecting signals generated by the plurality of conjugates. It should be understood that, in this case, signals generated by any one type of the plurality of conjugates are distinguishable from signals generated by other conjugates.

In a second implementation, the candidate aptamers are contacted with only one type of the targets at a time. In this case, step (b) includes: (b-22) introducing a first fluid onto the surface of the solid carrier, where the first fluid includes a first target and the first target carries the detectable label; and (b-42) subjecting the surface of the solid carrier to a designated environment for a certain duration to allow any of the candidate aptamers thereon having selectivity for the first target to conjugate with the first target to obtain a first conjugate; and step (c) includes: detecting a signal generated by the first conjugate; replacing the first fluid with an Nth fluid containing an Nth target to obtain an Nth conjugate; replacing the first conjugate with the Nth conjugate, where step (b) and step (c) are repeated, and N is a natural number greater than or equal to 2. In this implementation, the detectable labels carried by the plurality of targets may be the same or different.

In this implementation, each execution of steps (b-22), (b-42), and (c) constitutes one cycle, which is used to screen for an aptamer having selectivity for one type of the targets. When screening for a second type of candidate aptamer, steps (b-22), (b-42), and (c) are repeated. In this embodiment, before repeating step (b) and step (c), the method further includes removing the free target.

Illustratively, when two types of targets are used for aptamer screening, step (b) and step (c) include:
(b-222) introducing a first fluid onto the surface of the solid carrier after (a), where the first fluid includes a first target, and the first target carries the detectable label;
(b-422) subjecting the surface of the solid carrier to a designated environment for a certain duration to allow any of the candidate aptamers thereon having selectivity for the first target to conjugate with the first target to obtain a first conjugate;
step (c2) includes: detecting a signal generated by the first conjugate;
removing the free first target;
(b-224) introducing a second fluid onto the surface of the solid carrier, where the second fluid includes a second target, and the second target carries the detectable label;
(b-424) subjecting the surface of the solid carrier to a designated environment for a certain duration to allow any of the candidate aptamers thereon having selectivity for the second target to conjugate with the second target to obtain a second conjugate;
step (c4) includes: detecting a signal generated by the second conjugate.

The method for screening for an aptamer by sequencing according to the embodiments of the present application further includes:
(d) determining an aptamer having selectivity for the target based on the sequencing data and the signal.

By combining the sequencing data obtained in step (a) and the signals of the conjugates detected in step (c), the signals can be associated with the sequencing data through positional correlation, thereby determining the sequence of the aptamer associated with the conjugate that generates the signal, and further determining the sequence of the candidate aptamer having selectivity for the designated target.

In step (d), in combination with the sequencing data, the sequences of the candidate aptamers having selectivity for the designated target can be determined. In some embodiments, (d) includes: (d-10) processing images obtained from (c) to determine positions thereon of the emission signals corresponding to the conjugate; (d-30) registering the processed images in (d-10) obtained from (c) with the template to determine positions of overlapping emission signals; and (d-50) determining the sequence of the aptamer based on the reads corresponding to the positions of the overlapping emission signals. In an embodiment, (d-50) includes: aligning the reads with a reference sequence to determine positions of portions in the reads corresponding to the common sequences present at both ends of the candidate aptamer; and determining the sequence of the aptamer based on the positions and the reads.

In one implementation, step (a) includes step (a-20): separately constructing a sequencing library for each of the candidate aptamers, and making the sequencing library for each of the candidate aptamers have a specific tag and an adapter;
step b) includes: contacting the candidate aptamers with a target in a liquid environment, and allowing any of the candidate aptamers having selectivity for the target to conjugate with the target to obtain a conjugate;
step (c) includes: imaging the surface of the solid carrier to collect emission signals to obtain an image;
step (d) includes:
demultiplexing the sequencing data obtained in step (a) based on the specific tags, and saving the sequencing data having the same specific tag into separate fq.gz files, where the sequencing data having the same specific tag includes sequencing data of the candidate aptamer library (forward strand reads) and sequencing data of reverse complementary sequences of the candidate aptamer library (reverse strand reads);
binarizing the image obtained in step (c) to obtain a binarized image and first positional information of the candidate aptamers suspected of binding to the target in the binarized image;
registering the binarized image with the template, and determining whether an optical signal exists within the eight-neighborhood of the position center of each candidate aptamer in the binarized image; if yes, determining that the candidate aptamer binds to the target, and outputting second positional information of all candidate aptamers bound to the target;
searching for corresponding sequences in the demultiplexed fq.gz files based on the second positional information, and outputting the sequences into an fq format file;
aligning all sequences in the fq format file with the upstream and downstream sequences of the library and their reverse complementary sequences, and within a preset error tolerance range, obtaining the sequences that are aligned and outputting them into an fq format file; and
removing the adapters and the specific tags in the sequences to obtain the final sequences of the aptamers.

In this method, the candidate aptamers are subjected to conventional sequencing, and then targets carrying optical labels are introduced, followed by imaging of the candidate aptamers to obtain images, thereby facilitating the accurate screening for sequences of the aptamers bound to targets.

In another implementation, step (a) includes step (a-20): separately constructing a sequencing library for each of the candidate aptamers, and making the sequencing library for each of the candidate aptamers have a specific tag and an adapter; performing sequencing on the sequencing libraries, determining the sequences of the candidate aptamers and the positions of the sequences, and continuously obtaining sequencing data;
step (b) includes: contacting the candidate aptamers with one type of target in a liquid environment, and allowing any of the candidate aptamers having selectivity for the target to conjugate with the target to obtain a conjugate;
step (c) includes: imaging the surface of the solid carrier to collect emission signals to obtain an image;
repeating the operations of step (b) and step (c) based on the number of targets to be tested, while continuously obtaining sequencing data using base-calling software, automatically registering images obtained from step (c) at each time, extracting image intensities at all positions, and outputting them to a hard disk;
step (d) includes:
demultiplexing the sequencing data obtained based on the specific tags, and saving the sequencing data having the same specific tag into separate fq.gz files, where the sequencing data having the same specific tag includes sequencing data of the candidate aptamers (forward strand reads) and sequencing data of complementary sequences of the subsequent aptamers (reverse strand reads);
for all sequences in the fq.gz files, obtaining the signal intensity at the position of the sequence from step (c), and determining whether the signal intensity is greater than the preset signal intensity; if yes, determining that the sequence at that position as the sequence of the aptamer and outputting the sequence into an Fq file;
aligning all sequences in the fq format file with the upstream and downstream sequences of the library and their reverse complementary sequences, and within a preset error tolerance range, obtaining the sequences that are aligned and outputting them into an fq format file; and
removing the adapters and the specific tags in the sequences to obtain the final sequences of the aptamers.

In the above steps, by aligning all sequences in the fq format file with the upstream and downstream sequences of the library, the sequences of the aptamers with accurate upstream and downstream primers are screened, and the lengths of the sequences of the aptamers are determined. Thereafter, the sequences of the candidate aptamers that do not meet the requirements, such as those with inaccurate upstream and downstream primers, are removed and not used for subsequent analysis. In some embodiments, the preset error tolerance range is 1-3 bases.

In step (d), in combination with the sequencing data and the signal, the sequences of the candidate aptamers having selectivity for the designated target and the selectivity strength of the candidate aptamers for the designated target can be determined. In one scenario, one type of designated target simultaneously binds to two or more candidate aptamers, and, in combination with the sequencing data obtained in step (a), the sequences of the two or more candidate aptamers can be determined; in combination with the signals detected in step (c), the difference in the binding ability of the two types of candidate aptamers when binding to the designated target can be determined, thereby determining the aptamer having selectivity for the designated target. In addition, the nucleic acid aptamer that binds to the designated target in greater quantity is considered as the more selective aptamer. In another scenario, one type of candidate aptamer simultaneously binds to two or more different targets, and, in combination with the sequencing data obtained in step (a), the sequence of the candidate aptamer can be determined; in combination with the signals detected in step (c), the difference in the binding ability of the nucleic acid aptamer when binding to different targets can be determined. In addition, the nucleic acid aptamer is considered as more selective for the target to which it binds in greater quantity.

In some implementations, step (b) includes: (b-22) introducing a first fluid onto the surface of the solid carrier, where the first fluid includes a first target and the first target carries the detectable label; and (b-42) subjecting the surface of the solid carrier to a designated environment for a certain duration to allow any of the candidate aptamers thereon having selectivity for the first target to conjugate with the first target to obtain a first conjugate. Step (c) includes: detecting a signal generated by the first conjugate. In this case, (d) includes (d-22) analyzing the sequencing data and the signal generated by the first conjugate to identify an aptamer having selectivity for the first target. In some implementations, the method for screening for an aptamer by sequencing further includes: replacing the first fluid with an Nth fluid containing an Nth target to obtain an Nth conjugate; and replacing the first conjugate with the Nth conjugate, where step (b) and step (c) are repeated, and N is a natural number greater than or equal to 2. In this case, (d) further includes: (d-24) analyzing the sequencing data and the signal generated by the Nth conjugate to identify an aptamer having selectivity for the Nth target.

The present application further provides a reagent kit for screening for a nucleic acid aptamer. The reagent kit includes a solid carrier, a candidate aptamer, a target, and a reagent. The reagent includes a first reagent for use in attaching the candidate aptamer onto the surface of the solid carrier. In some embodiments, the reagent further includes a second reagent for use in sequencing a plurality of candidate aptamers attached to the surface of the solid carrier to obtain sequencing data. In some embodiments, the reagent further includes a third reagent for use in lysing the target. The reagent kit further includes a balance library. In some embodiments, the reagent kit includes a candidate aptamer solution, and the target solution includes the candidate aptamer, the balance library, and the first reagent. The selection of the candidate aptamer, the target, and the balance library is as described above. For example, in some embodiments, the candidate aptamer is a single-stranded nucleic acid molecule; in some embodiments, the candidate nucleic acid aptamer has a length of 20-150 nt. In some embodiments, the target is selected from at least one of proteins, viruses, bacteria, cells, and heavy metal ions; in some embodiments, the target is selected from at least two of streptavidin (SA), interleukin 3 receptor a (3RA), and colony stimulating factor (CSF). In some embodiments, the proportion of the balance library is not less than 10% of the total content of the candidate aptamers and the balance library; in some embodiments, the balance library is selected from microbial libraries having a GC content of 40%-50%; in some embodiments, the balance library is selected from at least one of E. coli 8 and PhiX174.

The feasibility of the method provided in the present application is verified and illustrated below with reference to specific embodiments.

### Verification Example

(1) A nucleic acid aptamer having selectivity for streptavidin (SA) was obtained, with a known sequence (SEQ ID NO:1) of the nucleic acid aptamer being:

AGCAGCACAGAGGTCAGATGGACGCACCGATCGCAGGTTCCGAAATGACT
ACTTGGTTGCCTATGCGTGCTACCGTGAA;

(2) The nucleic acid aptamer was subjected to PCR amplification to obtain a sequencing library including the above nucleic acid aptamer. A tag sequence (ATCACG) was added to both ends of the nucleic acid aptamer sequence (tagged aptamer; SEQ ID NO:2). Considering that the nucleic acid aptamer sequence is a single sequence, which may cause focusing and base-calling problems during sequencing imaging, a spike-in library was incorporated into the sequencing library to achieve sequence diversity. The spike-in library was an E. coli 8 library that does not bind to SA. Four parallel test groups were established by varying the proportion of the spike-in library to be added. The spike-in library contents in the four parallel test groups are as shown in Table 1.

TABLE 1

| NUMBER | LIBRARY INFORMATION | REMARKS |
|---|---|---|
| 1 | 100% nucleic acid aptamer library | Without |
| | | sequencing |
| 2 | Mixed library of 30% nucleic acid aptamer | Sequencing |
| | library and 70% E. coli 8 | |
| 3 | Mixed library of 10% nucleic acid aptamer | Sequencing |
| | library and 90% E. coli 8 | |
| 4 | Library of unknown nucleic acid sequences | Sequencing |
| | binding to SA | |

(3) The sequencing library was attached onto the surface of the solid carrier, and the sequencing library was amplified to obtain a clonal cluster. In this case, on the surface of the solid carrier, two types of clonal clusters formed from amplification of the nucleic acid aptamer existed: one being the first clonal cluster formed by DNA having the same sequence as the nucleic acid aptamer, and the other being the second clonal cluster formed by DNA reverse complementary to the nucleic acid aptamer sequence.

(4) The clonal cluster was sequenced to obtain sequencing data.

(5) Fluorescently labeled SA-alexa647 (labeled with alexa647) at a concentration of 1 μmol/L was introduced onto the surface of the solid carrier and allowed to react continuously for 30 minutes. Subsequently, 300 μL of PBST (phosphate-buffered saline with Tween) was used to flush the channel to remove unreacted fluorescently labeled SA-alexa647.

(6) A 640 nm excitation light was used to excite the fluorophore alexa647 to emit light, and the surface of the solid carrier was imaged to collect the corresponding emission signals. (The experimental group 1, including only the nucleic acid aptamer library, was not subjected to sequencing.) The sequencing results of experimental groups 2-4 are shown in Table 2 below.

TABLE 2

| | Total | | | | Actual |
|---|---|---|---|---|---|
| | throughput | Number of | | | effective |
| | (TOTAL- | templates | Cluster | | throughput |
| | READS) | (TEMPLATE | density | | (OUTPUT |
| Number | (K) | NUMPERFOV) | (μm²) | Q30 | READS (K) |
| 2 | 10126.56 | 112517 | 0.1791 | 87.8984 | 9035.46 |
| 3 | 8886.526 | 98739 | 0.1571 | 88.6781 | 8125.528 |
| 4 | 2601.438 | 28904 | 0.046 | 74.6794 | 2048.156 |

The sequencing Q30 of experimental groups 2 and 3 was approximately 88%. Even for experimental group 4, which did not contain a balance library, the sequencing Q30 approached 75%. Thus, the sequencing results of this example exhibited high accuracy.

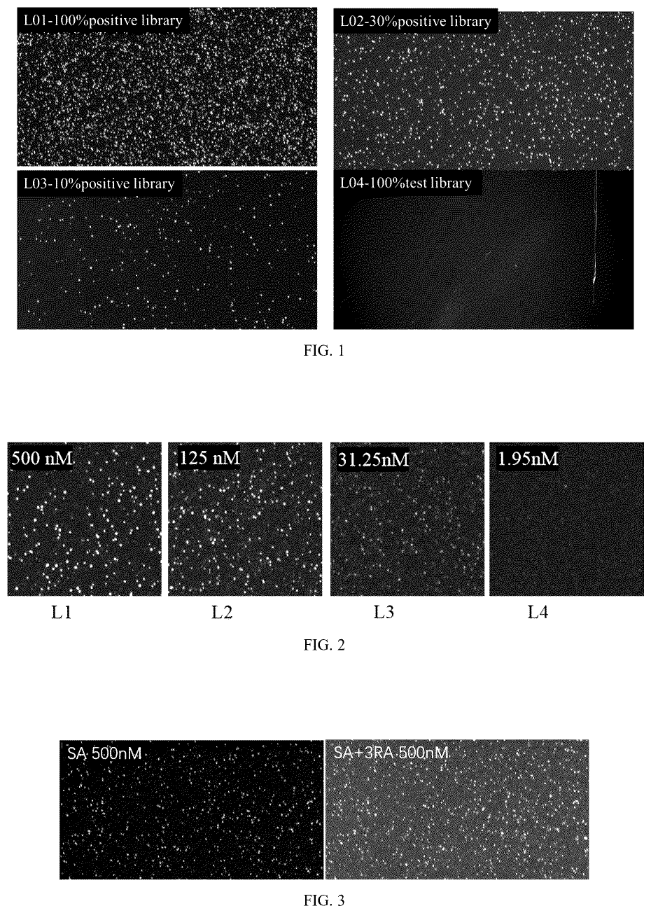

Imaging was performed for the four experimental groups, with the captured images shown in FIG. 1. L01, L02, L03, and L04 represent experimental groups 1-4, respectively. It can be seen from FIG. 1 that, with decreasing content of the positive library (SA nucleic acid aptamer), the number of fluorescence signal spots in the captured images decreased accordingly, and there was an obvious gradient relationship between the number of fluorescence signal spots and the content of the positive library (SA nucleic acid aptamer). The gradient relationship indicated that the binding of SA-alexa647 on the surface of the solid carrier was specific binding, rather than adsorption or other non-specific binding. In experimental group 4, the positive binding points of the unknown library were relatively few. Based on the morphology and intensity of the signals, it was inferred that an extremely small portion of strong-binding sequences was present. However, since no internal reference points were available, image recognition could not be performed.

The numbers of signal spots of SA-alexa647 (i.e., the number of conjugates formed between SA and the SA nucleic acid aptamer) in experimental groups 1-3 were counted, with the results shown in Table 3 below.

TABLE 3

| | POSITIVE | | SA-alexa647 | | |
|---|---|---|---|---|---|
| | LIBRARY | TOTAL | NUMBER OF | THEORETICAL | ACTUAL |
| | ADDITION | NUMBER OF | BINDING | BINDING | BINDING |
| Number | RATIO | TEMPLATES | SIGNAL SPOTS | RATIO | RATIO |
| 1 | 100% | 9328 | 4394 | 100% | 47.11% |
| 2 | 30% | 13338 | 1353 | 30%*47.11% = | 10.14% |
| | | | | 14.1% | |
| 3 | 10% | 12062 | 434 | 10%*47.11% = | 3.60% |
| | | | | 4.7% | |

It can be seen from Table 3 that the binding ratio in experimental group 1 was 47%, indicating that the SA nucleic acid aptamer and its complementary strand each accounted for approximately 50%, which was consistent with theoretical expectations. According to the statistical results, in experimental groups 2 and 3, the actual binding ratios were 10.14% and 3.60%, respectively, which were also close to the theoretical binding ratios of 14.1% and 4.7%. Thus, the number of fluorescence signal spots excited at 640 nm exhibited a gradient response to the positive library addition ratio. This concentration-dependent relationship demonstrated that the fluorescence signals originated from the specific binding of SA-alexa647 to the positive sequence, and that the number of SA molecules bound correlated positively with the content of the positive library. Accordingly, the method according to the embodiments of the present application can achieve highly accurate screening results by reflecting a favorable positive correlation between the binding ability and the binding frequency of aptamers with target molecules.

The sequencing results of the SA-positive sequence were obtained by demultiplexing based on the tag sequence. These results included forward strand reads (SA-positive sequence) and reverse strand reads (complementary strand of the SA-positive sequence as read by sequencing). The sequencing data at overlapping positions of SA-alexa647 binding images and positive sequence signals were outputted, namely, data of SA target-bound amplification clusters. The overlapping sequences of the sequences of the target-bound amplification clusters and the sequence of the SA-positive sequence demultiplexed based on the tag sequence were then counted.

The data analysis results for experimental groups 2 and 3 are shown in Table 4 and Table 5 below, respectively.

TABLE 4

| | Total | Reverse | Forward | |
|---|---|---|---|---|
| No. 2 (30% mix) | reads | strand reads | strand reads | Others |
| SA positive | 8956 | 4199 | 3875 | 882 |
| sequencing | | | | |
| Target-bound | 2808 | 0 | 2689 | 119 |
| amplification | | | | |
| cluster | | | | |

TABLE 5

| | Total | Reverse | Forward | |
|---|---|---|---|---|
| No. 3 (10% mix) | reads | strand reads | strand reads | Others |
| SA positive | 2334 | 1128 | 965 | 241 |
| sequencing | | | | |
| Target-bound | 575 | 2 | 558 | 15 |
| amplification | | | | |
| cluster | | | | |

It can be seen from Table 4 that, for experimental group 2, 69.39% (2689/3875) of the forward strand reads overlapped with the fluorescent points, while no reverse strand reads overlapped with the fluorescent points, indicating that the forward strand reads were the SA-positive sequence. Thus, nearly 70% of the forward strand amplification clusters could bind to the target, while no reverse strand overlapped, which was consistent with expectations of specific binding signal. Through algorithmic alignment, 2538 completely matched sequences were identified from 3875 reads. Among these 2538 sequences, 2059 reads overlapped with the fluorescent point positions, accounting for 81.13%. This result indicated that the SA target and the SA-positive sequence exhibited characteristics of specific binding.

It can be seen from Table 5 that, for experimental group 3, 57.82% (558/965) of the forward strand reads overlapped with the fluorescent points, while only 2 reverse strand reads overlapped with the fluorescent points. Thus, nearly 60% of the forward strand amplification clusters could bind to the target, which was consistent with expectations of specific binding signal. Through algorithmic alignment, among the completely matched sequences identified from 558 reads, 70.71% of the reads overlapped with the fluorescent point positions. This result indicated that the SA target and the SA-positive sequence exhibited characteristics of specific binding.

In addition, it can also be seen from Table 5 that when the content of positive sequence in the mixture library was 10%, the recognition and alignment of protein-binding fluorescence signals with sequencing signals could be achieved.

### Example 1

In this example, sequencing was performed on two types of nucleic acid aptamers targeting streptavidin (SA), i.e., a known positive sequence (SA-positive sequence) and a test positive sequence (test SA-P1 sequence), and in situ hybridization on the sequencing flowcell was performed to verify the feasibility of the experimental scheme and to screen for the appropriate concentration of SA protein for in situ hybridization on the surface of the sequencing flowcell.

The experimental setup was as follows:

(1) A known positive nucleic acid aptamer (i.e., SA-positive sequence) was obtained, having the sequence (SEQ ID NO: 1):

AGCAGCACAGAGGTCAGATGGACGCACCGATCGCAGGTTCCGAAATGACT
ACTTGGTTGCCTATGCGTGCTACCGTGAA.

A mixture library containing the SA-positive sequence and the test SA-P1 sequence was constructed, and the ratio of the SA-positive sequence to the test SA-P1 sequence was 1:1. A tag sequence: GGCTAC was added to the SA-positive sequence (tagged aptamer, SEQ ID NO: 3). Considering that the positive sequence is a single sequence, which may cause focusing and base-calling problems during sequencing imaging, 70% of the balance library PhiX174 was spiked into the positive sequence to achieve sequence diversity.

(2) The nucleic acid library was attached onto the surface of the solid carrier, and the sequencing library was amplified to obtain a clonal cluster. The clonal cluster was sequenced to obtain sequencing data of the three libraries.

(3) Into the four flow channels of the sequencing flowcell (L1, L2, L3, and L4 respectively), SA-alexa647 at concentrations of 500 nmol/L, 125 nmol/L, 31.25 nmol/L, and 1.95 nmol/L was introduced, respectively, and the flowcell was incubated for 30 minutes for in situ hybridization. After incubation, 300 μL of PBST was used to wash the channels to remove unreacted fluorescently labeled SA-alexa647.

(4) A 640 nm laser was used to excite the alexa647 to emit light, and the surface of the solid carrier was imaged to collect the corresponding emission signals.

The imaging results are shown in FIG. 2. It can be seen from FIG. 2 that the binding amount of SA-alexa647 to the positive library exhibited an obvious gradient relationship with the concentration of SA-alexa647, which further indicated that the positive sequence bound to SA could specifically bind to SA. In addition, as can be seen from FIG. 2, when the concentration was lower than 31 nmol/L, the binding signal of SA to the positive sequence was weak.

### Example 2

This example provides a method for screening for an aptamer by sequencing. The method includes the following steps:

(1) A mixture library containing three unknown sequence libraries and a balance library PhiX174 was constructed, and tag sequences TTAGGC, ATCACG, and CGATGT were respectively added to the three unknown sequence libraries. The content of the balance library PhiX174 was 40%, and the content ratio of the three unknown sequence libraries was 1:1:1.

(2) The nucleic acid library was attached onto the surface of the solid carrier, and the sequencing library was amplified to obtain a clonal cluster. The clonal cluster was sequenced to obtain sequencing data. The three libraries were demultiplexed based on their respective tag sequences, and the demultiplexing proportions, i.e., the obtained proportions, of the three libraries were close to the mixing ratio, indicating that the amplification efficiency of each library into clusters was normal.

(3) After sequencing was completed, target protein SA at a concentration of 500 nmol/L and a combination of target proteins SA and 3RA at 500 nmol/L each were sequentially introduced into the flowcell, followed by incubation. After each incubation was completed, 300 μL of PBST was used to wash the channels. After washing was completed, a 640 nm laser was used for excitation, and the surface of the solid carrier was imaged to collect the corresponding emission signals. Before introducing the next target at each time, the channels were washed with 750 μL of NaOH to remove the previously bound target.

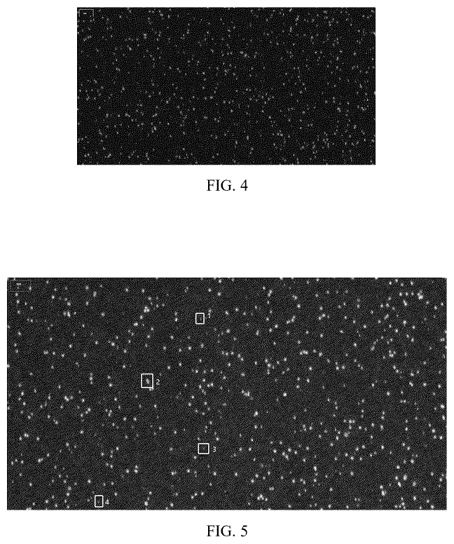

The imaging results are shown in FIG. 3. The images obtained after incubation with the target protein SA and with the combination of target proteins SA and 3RA were merged to generate FIG. 4, in which orange points represent the overlap between the combination of target proteins SA and 3RA and the target protein SA group, and green points represent amplification clusters that specifically bound to the target protein 3RA.

When screening for aptamers against target SA and the combination of targets SA and 3RA (i.e., a combined target containing both SA and 3RA targets), the fluorescence signal results were marked. Specifically, when different targets were introduced, the display of fluorescence signals included two cases: (1) fluorescence signal displayed, marked as Y; and (2) no fluorescence signal displayed, marked as N. Accordingly, for clonal clusters formed by the test SA-binding library, fluorescence signals were collected when either target SA or the combined target of SA and 3RA was introduced (marked as YY). For clonal clusters formed by the test 3RA-binding library, no fluorescence signals were collected when target SA was introduced (marked as N); however, fluorescence signals were collected when the combined target of SA and 3RA was introduced (marked as Y, which was finally marked as NY). For clonal clusters formed by the test PhiX174 library, no fluorescence signals were collected when either target SA or the combined target of SA and 3RA was introduced (marked as NN). Based on this, aptamers against the target SA and the target 3RA can be screened out separately. Using this method, the fluorescence image (FIG. 4) of the target SA and the combination of targets SA and 3RA (i.e., a combined target containing both SA and 3RA targets) was marked, and the results are shown in FIG. 5. The green points represent NY points, indicating that the library specifically bound to 3RA, and the yellow points represent YY points, indicating that the library bound to SA.

By applying the same method as in Example 2 to screen nucleic acid aptamers against 3RA and SA, four positive sequences binding to 3RA and one positive sequence binding to SA were obtained by screening, respectively, which are as follows:

3RA positive aptamer sequence 1 (SEQ ID NO: 4):

TCACGGTAGCACGCATAGGGGAGGATTCCGACTAAGTTTCCGTCGCATGA
GCGTTGCTATGCGTGGAGTGCTGAAGTTGGC

3RA positive aptamer sequence 2 (SEQ ID NO: 5):

CGAAGTTCAGCACTCCACGCATAGCCCTCGGAGGTCTACGAGAATGCCTA
CAGCCCTGTGCCCTATGCGTGCTACCGTGAA

3RA positive aptamer sequence 3 (SEQ ID NO: 6):

CACGGTAGCACGCATAACGCCGAGTTCCATTATAAAGTCCGCGCCCGACT
ACCCCCGTTATGCGTGGAGTGCTGGACATGC

3RA positive aptamer sequence 4 (SEQ ID NO: 7):

AATGGTCCAGCACTCCACGCATAACGAATAGGTCCTAAACGAGGGGTCAG
GGAAGTCGCCGGTTATGCGTGCTACCGTGAA

The SA positive aptamer sequence (SEO ID NO: 8) is as follows:

CTGGAAGTGACCTGCTATGGATTCAGATCGAGCAACACGAGCGTCTTAGT
ATCCTGTCACTTCAAGGCACCGTAGAGATCG

Based on the above examples, it can be concluded that the method for screening for an aptamer by sequencing according to the embodiments of the present application enables the determination of the base sequence of a test nucleic acid aptamer by screening and localizing in situ hybridization signals. By detecting the binding of targets to clonal clusters of known sequences on the sequencing flowcell, both the sequence information of the nucleic acid aptamer and the target-binding information can be obtained simultaneously, thereby completing aptamer screening. Since the number of clonal clusters on the surface of the sequencing flowcell may be extremely large, such as hundreds or even more, depending on factors such as the surface dimensions of the sequencing flowcell, this method can achieve high-throughput screening and theoretically detect a large number of nucleic acid sequences simultaneously. On this basis, a plurality of different targets can be screened simultaneously, thereby obtaining nucleic acid aptamers against a plurality of targets by screening. In addition, this method can also determine the binding strength between a target and a binding sequence via concentration titration.

In the description of the specification, references to the terms such as “one embodiment”, “some embodiments”, “schematic embodiments”, “examples”, “specific examples”, or “some examples” mean that the specific feature, structure, material, or characteristic described in connection with the embodiment or example is included in at least one embodiment or example of the present disclosure. In the specification, the schematic description of the above terms does not necessarily refer to the same embodiment or example. Moreover, the particular feature, structure, material, or characteristic described may be combined in any one or more embodiments or examples in any appropriate manner.

Although the embodiments of the present disclosure have been illustrated and described above, it will be appreciated that the aforementioned embodiments are exemplary and should not be construed as limiting the present disclosure, and that those of ordinary skill in the art can make changes, modifications, replacements, and variations to such embodiments, without departing from the scope of the present disclosure.

What is claimed is:

1. A method for screening for an aptamer by sequencing, comprising:
(a) sequencing a plurality of candidate aptamers attached to a surface of a solid carrier to obtain sequencing data;
(b) contacting the candidate aptamers with a target in a liquid environment, and allowing any of the candidate aptamers thereon having selectivity for the target to conjugate with the target to obtain a conjugate;
(c) detecting a signal generated by the conjugate; and
(d) determining an aptamer having selectivity for the target based on the sequencing data and the signal.

2. The method according to claim 1, wherein the candidate aptamers are single-stranded nucleic acid molecules, and the candidate aptamers have a length of 20-150 nt.

3. The method according to claim 2, wherein (a) comprises:
(a-10) obtaining a sequencing library comprising the candidate aptamers;
(a-30) attaching the sequencing library onto the surface of the solid carrier;
(a-50) amplifying the sequencing library to obtain a clonal cluster; and
(a-70) sequencing the clonal cluster to obtain the sequencing data.

4. The method according to claim 2, wherein (a) comprises:
(a-22) separately constructing a sequencing library for each of the candidate aptamers, comprising making the sequencing library for each of the candidate aptamers have a specific tag and an adapter;
(a-42) performing rolling circle amplification on the sequencing libraries for each of the candidate aptamers to form nanoballs of each of the candidate aptamers;
(a-43) mixing the nanoballs of the plurality of candidate aptamers according to a predetermined ratio;
(a-62) attaching the mixed nanoballs onto the surface of the solid carrier; and
(a-80) sequencing the nanoballs to obtain the sequencing data.

5. The method according to claim 3, wherein the sequencing is sequencing by synthesis, comprising: introducing a fluid containing a sequencing primer onto the surface of the solid carrier and subjecting the surface of the solid carrier to conditions suitable for a polymerization reaction, so as to controllably extend at least a portion of the sequencing primer and correspondingly detect an extension signal, wherein the sequencing primer is capable of matching at least a portion of the adapter.

6. The method according to claim 5, further comprising removing the sequencing primer or an extended strand formed by the sequencing primer before (b).

7. The method according to claim 1, wherein (b) comprises:
(b-20) introducing a target-containing fluid onto the surface of the solid carrier after (a), the target-containing fluid comprising one or more types of targets, and the target carrying a detectable label; and
(b-40) subjecting the surface of the solid carrier to a designated environment for a certain duration to allow any of the candidate aptamers thereon having selectivity for the target to conjugate with the target to obtain the conjugate.

8. The method according to claim 7, further comprising removing a free target after (b) or before (c).

9. The method according to claim 8, wherein (b) comprises:
(b-22) introducing a first fluid onto the surface of the solid carrier, the first fluid comprising a first target, and the first target carrying the detectable label;
(b-42) subjecting the surface of the solid carrier to a designated environment for a certain duration to allow any of the candidate aptamers thereon having selectivity for the first target to conjugate with the first target to obtain a first conjugate; and
(c) comprises: detecting a signal generated by the first conjugate,
replacing the first fluid with an Nth fluid containing an Nth target to obtain an Nth conjugate; replacing the first conjugate with the Nth conjugate, wherein step (b) and step (c) are repeated, and N is a natural number greater than or equal to 2.

10. The method according to claim 9, further comprising, before repeating step (b) and step (c),
removing the free target, and wherein step (d) comprises:
(d-22) analyzing the sequencing data and the signal generated by the first conjugate to identify an aptamer having selectivity for the first target; and/or
(d-24) analyzing the sequencing data and the signal generated by the Nth conjugate to identify an aptamer having selectivity for the Nth target.

11. The method according to claim 1, wherein the detectable label is a fluorophore, and (c) comprises exciting the fluorophore to emit light and imaging the surface of the solid carrier to collect emission signals.

12. The method according to claim 7, wherein the target-containing fluid comprises a plurality of targets, and a signal generated by a detectable label carried by any one type of the targets is distinguishable from a signal generated by a detectable label carried by other targets.

13. The method according to claim 7, wherein the plurality of targets comprised in the target-containing fluid are selected from at least two of streptavidin, interleukin 3 receptor a, colony stimulating factor, recombinant human PDGF-BB, MUC1, and human epidermal growth factor receptor-2.

14. The method according to claim 13, wherein the detectable label is a fluorophore, the plurality of targets comprised in the target-containing fluid each carry different fluorophores, and (c) comprises separately exciting the different fluorophores to emit light and imaging the surface of the solid carrier to collect corresponding emission signals.

15. The method according to claim 1, wherein a concentration of the target in a solution or a fluid containing the target is not less than 30 nmol/L.

16. The method according to claim 4, wherein in (a), the sequencing is sequencing by synthesis based on microscopic fluorescence imaging, performing corresponding extension signal detection comprises exciting the fluorescent label on the surface of the solid carrier after extension to emit light and capturing images, and obtaining the sequencing data comprises:
constructing a template based on a plurality of images obtained by capturing images, the template being a set of specific light-emitting positions on the plurality of images, and the set of specific light-emitting positions corresponding to positional information of the plurality of candidate aptamers on the surface of the solid carrier,
identifying types of extended bases based on the images and the template to obtain a plurality of reads, and
demultiplexing the reads based on portions of the reads corresponding to the specific tags to obtain the sequencing data of each of the candidate aptamers.

17. The method according to claim 16, wherein (d) comprises:
(d-10) processing images obtained from (c) to determine positions thereon of the emission signals corresponding to the conjugate;
(d-30) registering the processed images in (d-10) obtained from (c) with the template to determine positions of overlapping emission signals; and
(d-50) determining a sequence of the aptamer based on the reads corresponding to the positions of the overlapping emission signals.

18. The method according to claim 17, wherein (d-50) comprises:
aligning the reads with a reference sequence to determine positions of portions in the reads corresponding to the common sequences present at both ends of the candidate aptamer; and
determining the sequence of the aptamer based on the positions and the reads.


SEQUENCE LISTING (8 sequences)

SEQ ID NO: 1 - DNA, 79 bp, synthetic construct
  source 1..79: mol_type = other DNA
agcagcacagaggtcagatggacgcaccgatcgcaggttccgaaatgactacttggttgcctatgcgtgctaccgtgaa

SEQ ID NO: 2 - DNA, 91 bp, synthetic construct
  source 1..91: mol_type = other DNA
atcacgagcagcacagaggtcagatggacgcaccgatcgcaggttccgaaatgactacttggttgcctatgcgtgctaccgtgaaatcacg

SEQ ID NO: 3 - DNA, 91 bp, synthetic construct
  source 1..91: mol_type = other DNA
ggctacagcagcacagaggtcagatggacgcaccgatcgcaggttccgaaatgactacttggttgcctatgcgtgctaccgtgaaggctac

SEQ ID NO: 4 - DNA, 81 bp, synthetic construct
  source 1..81: mol_type = other DNA
tcacggtagcacgcataggggaggattccgactaagtttccgtcgcatgagcgttgctatgcgtggagtgctgaagttggc

SEQ ID NO: 5 - DNA, 81 bp, synthetic construct
  source 1..81: mol_type = other DNA
cgaagttcagcactccacgcatagccctcggaggtctacgagaatgcctacagccctgtgccctatgcgtgctaccgtgaa

SEQ ID NO: 6 - DNA, 81 bp, synthetic construct
  source 1..81: mol_type = other DNA
cacggtagcacgcataacgccgagttccattataaagtccgcgcccgactacccccgttatgcgtggagtgctggacatgc

SEQ ID NO: 7 - DNA, 81 bp, synthetic construct
  source 1..81: mol_type = other DNA
aatggtccagcactccacgcataacgaataggtcctaaacgaggggtcagggaagtcgccggttatgcgtgctaccgtgaa

SEQ ID NO: 8 - DNA, 81 bp, synthetic construct
  source 1..81: mol_type = other DNA
ctggaagtgacctgctatggattcagatcgagcaacacgagcgtcttagtatcctgtcacttcaaggcaccgtagagatcg